(19) United States
(12) Patent Application Publication
(10) Pub. No.: US 2021/0401431 A1
(43) Pub. Date: Dec. 30, 2021

(54) SURGICAL INSTRUMENT WITH FLEXIBLE DRIVE ROD

(71) Applicant: Covidien LP, Mansfield, MA (US)

(72) Inventors: Srinivasa Murthy Aravalli AVVLN, Hyderabad (IN); Ravi Sekhar Gutti, Hyderabad (IN); Jitendra Bhargava Srinivas, Hyderabad (IN); Suresh Kumar Prema Mohanasundaram, Chennai (IN); Raja Kamaraj, Hyderabad (IN); Rajanikanth Mandula, Hyderabad (IN)

(21) Appl. No.: 17/314,206

(22) Filed: May 7, 2021

Related U.S. Application Data
(60) Provisional application No. 63/045,232, filed on Jun. 29, 2020.

(51) Int. Cl. A61B 17/068; A61B 17/072

(52) U.S. Cl. CPC A61B 17/0686; A61B 2017/00367; A61B 17/072

(57) ABSTRACT

A surgical instrument includes a handle assembly, a shaft extending distally from the handle assembly, an articulating section coupled to the shaft, and an end effector coupled to the articulating section. The handle assembly includes a rotatable knob, a first lever, a second lever, and an actuating handle. A flexible drive rod extends through the shaft to the end effector. First and second articulation cables extend through the shaft and are coupled to the articulating section. A proximal end of the flexible drive rod is coupled to the actuating handle and a distal end of the flexible drive rod is coupled to the end effector. A proximal end of the first articulation cable is coupled to the first lever and a proximal end of the second articulation cable is coupled to the second lever. Distal ends of the first and second articulation cables are coupled to the articulating section.

### CROSS REFERENCE TO RELATED APPLICATION

This application claims the benefit of the filing date of provisional U.S. Application No. 63/045,232, filed on Jun. 29, 2020.

### FIELD

This disclosure is generally related to surgical instruments and, more particularly, to surgical instruments and components for surgical instruments that include a flexible drive rod configuration.

### BACKGROUND

Various types of surgical instruments used to surgically join tissue are known in the art, and are commonly used, for example, for closure of tissue or organs in transection, resection, anastomoses, for occlusion of organs in thoracic and abdominal procedures, and for mechanically sealing tissue.

One example of such a surgical instrument is a surgical stapling instrument, which may include an anvil assembly, a cartridge assembly for supporting an array of surgical staples, an approximation mechanism for approximating the cartridge and anvil assemblies, and a firing mechanism for ejecting the surgical staples from the cartridge assembly.

Typically, surgical instruments include rigid elongated shafts thereby limiting their surgical applications because of their inability to flex, bend, or conform to the shapes of organs in which the surgical instrument is placed. The rigid elongated shaft of the surgical instruments therefore limits the access to tissue during procedures conducted through natural orifices or during endoscopic procedures.

### SUMMARY

This disclosure is directed to surgical instruments that include a flexible drive rod and articulating end effector.

In accordance with aspects of the disclosure, a surgical instrument includes a handle assembly, a shaft extending distally from the handle assembly, an articulating section operably coupled to a distal end of the shaft, and an end effector operably coupled to the articulating section. The handle assembly includes a rotatable knob, a first lever, a second lever, and an actuating handle. A flexible drive rod extends through the shaft and is operably coupled to the end effector. A first articulation cable and a second articulation cable extend through the shaft and are operably coupled to the articulating section. A proximal end of the flexible drive rod is operably coupled to the actuating handle of the handle assembly and a distal end of the flexible drive rod is operably coupled to the end effector such that actuation of the actuating handle causes actuation of the flexible drive rod. The first articulation cable has a proximal end operably coupled to the first lever of the handle assembly and a distal end operably coupled to the articulating section. The second articulation cable has a proximal end operably coupled to the second lever of the handle assembly and a distal end operably coupled to the articulating section.

In aspects, the first lever is operably coupled to the second lever such that movement of the first lever in a first direction causes corresponding movement of the second lever is a second direction opposite the first direction.

In aspects, movement of at least one of the first lever or the second lever causes the articulating section to articulate.

In aspects, the articulating section includes a proximal articulation link and a distal articulation link. The proximal articulation link is coupled to the distal end of the shaft and the distal articulation link is coupled to the end effector. Additionally, or alternatively, the distal end of the first articulation cable and the distal end of the second articulation cable are operably coupled to the distal articulation link of the articulating section.

In aspects, at least one of the first articulation cable or the second articulation cable is lockable in position to prevent articulation of the articulating section.

In aspects, at least one of the first lever or the second lever is lockable in position to prevent articulation of the articulating section.

In aspects, the shaft is flexible and configured to bend when an external force is imparted thereon.

In aspects, the shaft is defined by a thin-walled tube having serrated cuts along its length configured to enable flexible motion of the shaft.

In aspects, the shaft is configured to be transanally introduced into an intestine of a patient and to deform to a shape of the intestine.

In aspects, the handle assembly further includes a fixed handle and the actuating handle is movable relative to the fixed handle.

In aspects, the handle assembly further includes a rotatable knob and the shaft is fixed to the rotatable knob such that rotation of the rotatable knob causes rotation of the shaft.

In aspects, the flexible drive rod is defined by a plurality of interlocking links. Each link of the plurality of interlocking links may define a dovetail shape to interlock with an adjacent link.

In aspects, the surgical instrument is at least one of a linear surgical stapler or a circular surgical stapler.

In aspects, the end effector includes a stapler cartridge housing surgical staples and an anvil movable relative to the stapler cartridge.

In another aspect of the disclosure, a surgical instrument includes a shaft having a distal end, an articulating section operably coupled to the distal end of the shaft, an end effector operably coupled to the articulating section, and a flexible drive rod extending through the shaft and the articulating section and having a distal end. The distal end of the flexible drive rod is operably coupled to the end effector for actuation of the end effector. The surgical instrument further includes a first articulation cable and a second articulation cable extending through the shaft. The first articulation cable has a distal end operably coupled to the articulating section and the second articulation cable has a distal end operably coupled to the articulating section.

In aspects, the flexible drive rod is defined by a plurality of interlocking links. Each link of the plurality of interlocking links may define a dovetail shape to interlock with an adjacent link.

In aspects, the surgical instrument is at least one of a linear surgical stapler or a circular surgical stapler.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

Various aspects of the disclosure are described herein below with reference to the drawings, wherein:

FIG. 1 is a side view of a surgical instrument including aspects of the disclosure;

FIG. 2 is a perspective view of a handle assembly of the surgical instrument of FIG. 1;

FIG. 3 is a perspective view of a portion of the surgical instrument of FIG. 1, including an articulating section, a portion of a drive rod, and a portion of a shaft, with parts removed;

FIG. 4 is a perspective view of a distal portion of the surgical instrument of FIG. 1, including an end effector, with parts removed;

FIG. 5 is perspective view of the surgical instrument of FIG. 1 with a shaft of the surgical instrument bent and an articulating section of the surgical instrument articulated;

FIG. 6 is an enlarged view of the indicated area of detail shown in FIG. 5 including the articulating section of the surgical instrument of FIG. 1; and

FIG. 7 is an enlarged view of the indicated area of detail shown in FIG. 5 including serrated cuts along a surface of the shaft of the surgical instrument of FIG. 1.

### DETAILED DESCRIPTION

In this description, the term “proximal” is used generally to refer to that portion of the device that is closer to a clinician, while the term “distal” is used generally to refer to that portion of the device that is farther from the clinician. Further, the term “clinician” is used generally to refer to medical personnel including doctors, nurses, and support personnel.

The disclosure describes a powered, or non-powered, surgical instruments, for example linear and circular staplers, that incorporate a flexible shaft and/or a flexible drive rod. The flexible nature of the components of the surgical instrument improves the accessibility of the surgical instrument to various anatomical locations and organs during endoscopic surgical procedures or surgical procedures conducted through natural body orifices. For example, the disclosed surgical instruments may be introduced into natural orifices for surgical applications including endo-luminal fundoplication, colectomies and anastomosis. The flexible shaft of the disclosed surgical instruments, when used in conjunction with a flexible drive rod, enable the surgical instrument to take the shape of the organ in which it is placed, for example an intestine when introduced transanally, thereby minimizing damage to the organ in which it is introduced while maintaining functionality of the instrument.

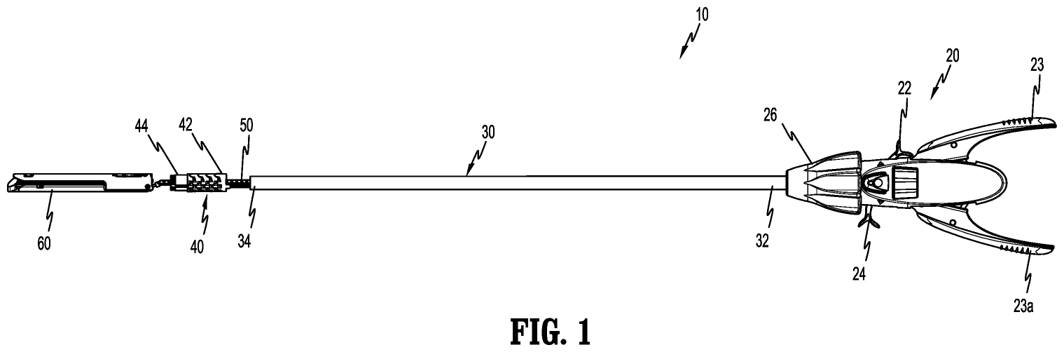

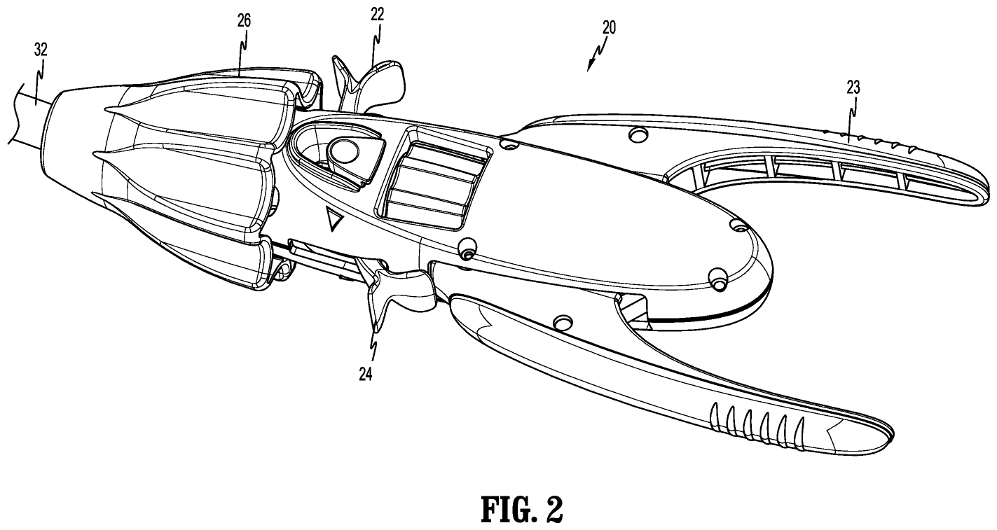

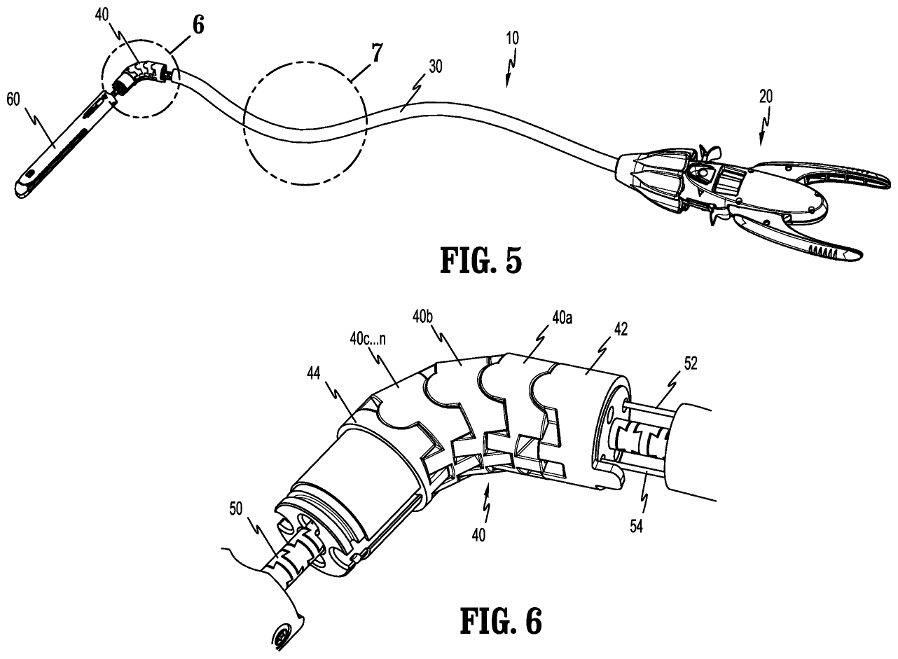

FIGS. 1 and 5 illustrate an exemplary surgical instrument 10 in accordance with the disclosure. The surgical instrument 10 includes a handle assembly 20, a shaft 30 extending distally from the handle assembly 20, an articulating section 40 operably coupled to a distal end 34 of the shaft 30, and an end effector 60 operably coupled to the articulating section 40. The handle assembly 20 includes a rotatable knob 26, a first lever 22, a second lever 24, and an actuating handle 23. The handle assembly 20 may further include a fixed handle 23a with the actuating handle 23 being movable relative to the fixed handle 23a.

The end effector 60 is articulatable relative to the shaft 30 via the articulating section 40. Additionally, the shaft 30 is fixedly coupled to the rotatable knob 26 of the handle assembly 20 such that rotation of the rotatable knob 26 causes rotation of the end effector 60.

A flexible drive rod 50 extends through the shaft 30 and is operably coupled to the end effector 60. In particular, a proximal end of the flexible drive rod 50 is operably coupled to the actuating handle 23 of the handle assembly 20 and a distal end of the flexible drive rod 50 is operably coupled to the end effector 60 such that actuation of the actuating handle 23 causes actuation of the flexible drive rod 50. When the actuating handle 23 is actuated, for example manually by a user or by a motor (not shown), the flexible drive rod 50 is driven linearly in a proximal or distal direction. The linear movement of the flexible drive rod 50 causes the jaws (e.g., stapler cartridge 62 and anvil 64 illustrated in FIG. 4) of the end effector 60 to transition between an open configuration and a closed configuration, and additionally, to fire staples from a stapler cartridge of the end effector 60. The flexible drive rod 50 may be defined by a plurality of interlocking links 50a, 50b, 50c, 50d . . . n, for example, with each link of the plurality of interlocking links 50a, 50b, 50c, 50d . . . n defining a dovetail shape to interlock with an adjacent link.

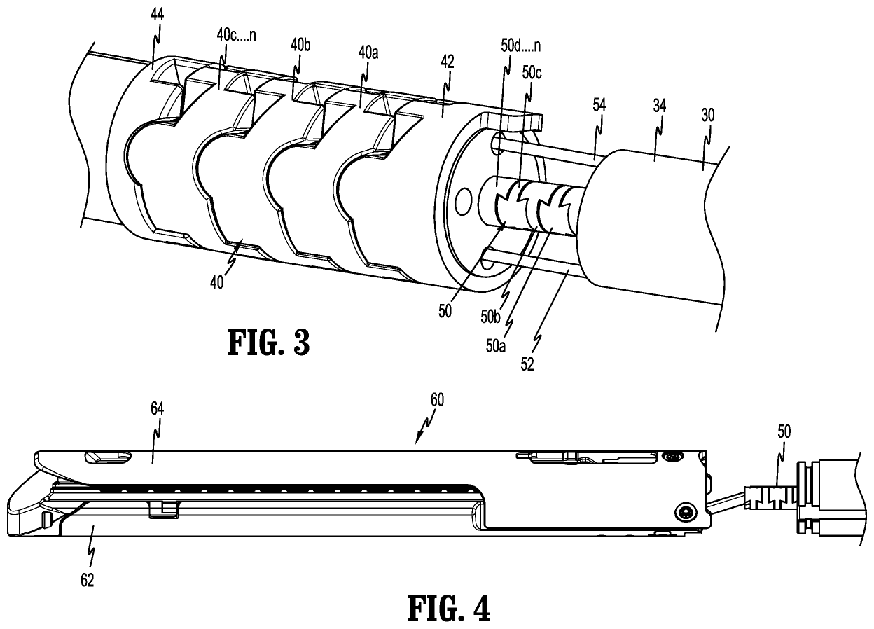

FIGS. 3, 5, and 6 illustrate a first articulation cable 52 and a second articulation cable 54 of the surgical instrument 10. The first articulation cable 52 and the second articulation cable 54 extend through the shaft 30 radially outward of the flexible drive rod 50 and are operably coupled to the articulating section 40, for example on opposing sides of the drive rod 50. The first articulation cable 52 has a proximal end operably coupled to the first lever 22 of the handle assembly 20 and a distal end operably coupled to the articulating section 40. Likewise, the second articulation cable 54 has a proximal end operably coupled to the second lever 24 of the handle assembly 20 and a distal end operably coupled to the articulating section 40. The first lever 22 is operably coupled to the second lever 24 about a pivot point such that movement of the first lever 22 in a first direction causes corresponding movement of the second lever 24 in a second direction opposite the first direction. Movement of at least one of the first lever 22 or the second lever 24 causes the articulating section 40 to articulate, and in turn, orients the end effector 60 relative to the shaft 30.

The articulating section 40 includes a proximal articulation link 42 and a distal articulation link 44 and may optionally include one or more intermediary links 40a, 40b, 40c . . . n between the proximal articulation link 42 and the distal articulation link 44. Although three intermediary links 40a, 40b, 40c are illustrated and described, it is appreciated that more or less than three links may be disposed between the proximal articulation link 42 and the distal articulation link 44. The proximal articulation link 42 is coupled to the distal end 34 of the shaft 30 and the distal articulation link 44 is coupled to the end effector 60. The distal end of the first articulation cable 52 and the distal end of the second articulation cable 54 are operably coupled to the distal articulation link 44 of the articulating section 40.

Components of the surgical instrument 10 may be lockable in position to prevent inadvertent movement or articulation of the end effector 60. For example, at least one of the first articulation cable 52 or the second articulation cable 54 may be lockable in position to prevent articulation of the articulating section 40. Additionally, or alternatively, at least one of the first lever 22 or the second lever is lockable in position to prevent articulation of the articulating section 40 including at least one or more of the links (e.g., proximal articulation link 42, distal articulation link 44, or intermediary links 40a, 40b, 40c . . . n) of the articulating section 40 from articulating or pivoting relative to each other.

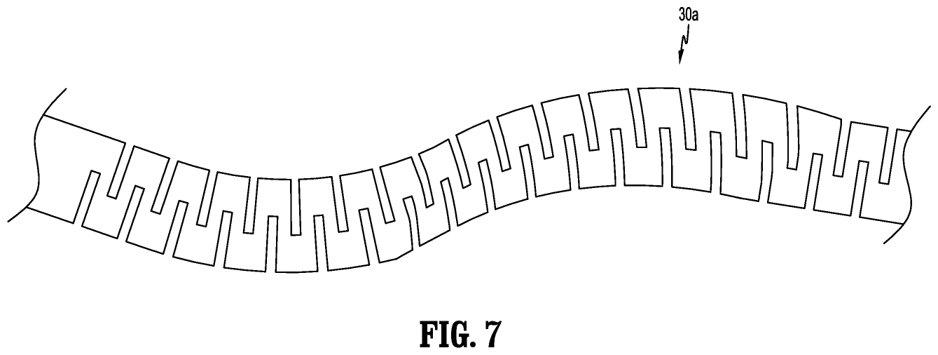

The shaft 30 is flexible and configured to bend when an external force is imparted thereon. For example, the natural shape of an organ in which the shaft 30 is placed may cause the shape of the shaft 30 to change (via bending or flexing) as the shaft 30 is introduced through the organ. In aspects, the shaft 30 is configured to be transanally introduced into an intestine of a patient and to deform to a shape of the intestine. The shaft 30 may be defined by a thin-walled tube having serrated cuts 30a (FIG. 7) along its length configured to enable flexible motion of the shaft 30.

The surgical instrument 10 is illustrated as a manually actuatable surgical instrument, but it is appreciated that surgical instrument 10 may be an electrically powered surgical instrument including an electrically powered handle assembly that may support one or more batteries (not shown). It is envisioned that the disclosed aspects could also be incorporated into a surgical instrument that is configured for use with a robotic system that does not include a handle assembly, or to a surgical instrument including a manually actuated handle assembly. Further, although illustrated and described as a surgical stapler instrument, it is envisioned that surgical instrument 10 may be any type of surgical instrument, such as a grasping forceps or other surgical instrument.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A surgical instrument comprising:
a handle assembly including a first lever, a second lever, and an actuating handle;
a shaft extending distally from the handle assembly and having a distal end;
an articulating section operably coupled to the distal end of the shaft;
an end effector operably coupled to the articulating section;
a flexible drive rod extending through the shaft and the articulating section and having a proximal end and a distal end, wherein the proximal end of the flexible drive rod is operably coupled to the actuating handle of the handle assembly and the distal end of the flexible drive rod is operably coupled to the end effector and wherein actuation of the actuating handle causes actuation of the flexible drive rod; and
a first articulation cable and a second articulation cable extending through the shaft, the first articulation cable having a proximal end operably coupled to the first lever of the handle assembly and a distal end operably coupled to the articulating section, the second articulation cable having a proximal end operably coupled to the second lever of the handle assembly and a distal end operably coupled to the articulating section.

2. The surgical instrument of claim 1, wherein the first lever is operably coupled to the second lever such that movement of the first lever in a first direction causes corresponding movement of the second lever is a second direction opposite the first direction.

3. The surgical instrument of claim 1, wherein movement of at least one of the first lever or the second lever causes the articulating section to articulate in relation to the shaft.

4. The surgical instrument of claim 1, wherein articulating section includes a proximal articulation link and a distal articulation link, the proximal articulation link coupled to the distal end of the shaft and the distal articulation link coupled to the end effector.

5. The surgical instrument of claim 4, wherein the distal end of the first articulation cable and the distal end of the second articulation cable are operably coupled to the distal articulation link of the articulating section.

6. The surgical instrument of claim 1, wherein at least one of the first articulation cable or the second articulation cable is lockable in position to prevent articulation of the articulating section.

7. The surgical instrument of claim 1, wherein at least one of the first lever or the second lever is lockable in position to prevent articulation of the articulating section.

8. The surgical instrument of claim 1, wherein the shaft is flexible and configured to bend when an external force is imparted thereon.

9. The surgical instrument of claim 1, wherein the shaft is defined by a thin-walled tube having serrated cuts along its length configured to enable flexible motion of the shaft.

10. The surgical instrument of claim 1, wherein the shaft is configured to be transanally introduced into an intestine of a patient and to deform to a shape of the intestine.

11. The surgical instrument of claim 1, wherein the handle assembly further includes a fixed handle and the actuating handle is movable relative to the fixed handle.

12. The surgical instrument of claim 1, wherein the handle assembly further includes a rotatable knob and the shaft is fixed to the rotatable knob such that rotation of the rotatable knob causes rotation of the shaft.

13. The surgical instrument of claim 1, wherein the flexible drive rod is defined by a plurality of interlocking links.

14. The surgical instrument of claim 13, wherein each link of the plurality of interlocking links defines a dovetail shape to interlock with an adjacent link.

15. The surgical instrument of claim 1, wherein the surgical instrument is at least one of a linear surgical stapler or a circular surgical stapler.

16. The surgical instrument of claim 1, wherein the end effector includes a stapler cartridge housing surgical staplers and an anvil movable relative to the stapler cartridge.

17. A surgical instrument comprising:
a shaft having a distal end;
an articulating section operably coupled to the distal end of the shaft;
an end effector operably coupled to the articulating section;
a flexible drive rod extending through the shaft and the articulating section and having a distal end, wherein the distal end of the flexible drive rod is operably coupled to the end effector for actuation of the end effector; and
a first articulation cable and a second articulation cable extending through the shaft, the first articulation cable having a distal end operably coupled to the articulating section, the second articulation cable having a distal end operably coupled to the articulating section.

18. The surgical instrument of claim 17, wherein the flexible drive rod is defined by a plurality of interlocking links.

19. The surgical instrument of claim 18, wherein each link of the plurality of interlocking links defines a dovetail shape to interlock with an adjacent link.

20. The surgical instrument of claim 1, wherein the surgical instrument is at least one of a linear surgical stapler or a circular surgical stapler.
